(19) United States
(12) Patent Application Publication
(10) Pub. No.: US 2007/0149451 A1
(43) Pub. Date: Jun. 28, 2007

(54) Combination of a dpp IV inhibitor and an antiobesity or appetite regulating agent

(76) Inventor: David Grenville Holmes, Binningen (CH)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/579,580

(22) PCT Filed: Nov. 11, 2004
(86) PCT No.: PCT/EP04/12989

§ 371 Date: Jan. 25, 2007

Related U.S. Application Data
(60) Provisional application No. 60/520,564, filed on Nov. 17, 2003.

(51) Int. Cl. A61K 38/17; A61K 31/4704; A61K 31/47; A61K 31/4439; A61K 31/40; A61K 31/137

(52) U.S. Cl. 514/12; 514/309; 514/310; 514/408; 514/343; 514/649

(57) ABSTRACT

The present invention relates to a combination, such as a combined preparation or pharmaceutical composition, respectively, comprising of a DPP IV inhibitor or a pharmaceutically acceptable salt thereof and an antiobesity agent, or an appetite regulating agent, or a pharmaceutically acceptable salt thereof. The present invention furthermore relates to the use of such a combination for the prevention, delay of progression or treatment of diseases and disorders selected from the group consisting of hypertension, congestive heart failure, left ventricular hypertrophy, peripheral arterial disease, diabetes, especially type 2 diabetes mellitus, diabetic retinopathy, macular degeneration, cataract, diabetic nephropathy, glomerulosclerosis, chronic renal failure, diabetic neuropathy, syndrome X, premenstrual syndrome, coronary heart disease, angina pectoris, thrombosis, atherosclerosis, myocardial infarction, transient ischemic attacks, stroke, vascular restenosis, hyperglycemia, hyperinsulinemia, hyperlipidemia, hypertryglyceridemia, insulin resistance, impaired glucose metabolism, conditions of impaired glucose tolerance, conditions of impaired fasting plasma glucose, obesity, erectile dysfunction, skin and connective tissue disorders, foot ulcerations and ulcerative colitis, endothelial dysfunction and impaired vascular compliance.

Obesity is a common and chronic condition whose prevalence has increased steadily in advanced nations. Though the molecular pathways regulating energy balance are beginning to be illuminated, the causes of obesity remain elusive. In part, this reflects the fact that obesity is a heterogeneous group of disorders. At one level, the pathophysiology of obesity seems simple: a chronic excess of nutrient intake relative to the level of energy expenditure. However, due to the complexity of the neuroendocrine and metabolic systems that regulate energy intake, storage, and expenditure, it has been difficult to quantitate all the relevant parameters (e.g., food intake and energy expenditure) over time in human subjects. Obesity contributes to a myriad of health problems, including type 2 diabetes mellitus, hypertension, congestive heart failure, lipid disorders, arthritis, and some cancers. Obesity and related conditions contribute to nearly 300 000 annual deaths in the United States. Unfortunately, obesity is not well understood.

Obesity is important as a risk factor of the onset of diseases represented by geriatric diseases from hygienic and cosmetic viewpoints. Harmful influences of obesity have been recognized for a long time in advanced nations. Agents for preventing and/or treating obesity which have been developed until now have side effects or produce unsatisfactory effects. Despite short-term benefits, medication-induced weight loss is often associated with rebound weight gain after the cessation of drug use, side effects from the medications, and the potential for drug abuse. Given the need for effective therapies, many possible compounds and combinations have been evaluated. For instance when fenfluramine was administered together with phentermine, as “fen-phen,” the combination was widely used based on controlled trials that demonstrated modest but definite efficacy. However, the risk of primary pulmonary hypertension was increased up to 20-fold in association with this treatment. The FDA withdrew approval of the fen-phen combination in 1997 when reports suggested an association with right- and left-sided valvular heart disease.

Thus there still exists a need for more effective anti-obesity combinations with less or no side effects such as described herein and lower toxicity.

Besides genetic predisposition, obesity is the most important risk factor for the development of type 2 diabetes mellitus. Even modest weight reduction can improve blood glucose control in overweight subjects. Thus, there is furthermore a need for a combination which is also effective for treating or preventing diabetes (or Impaired Glucose Tolerance (IGT)) with less or no side effects such as described herein e.g. weight gain and lower toxicity.

Particularly, there is a need of new combinations for the treatment and/or prevention of diabetes, IGT or obesity and showing furthermore beneficial effects on diseases and conditions associated with diabetes or obesity.

Therefore, an object of the present invention is to provide more effective anti-obesity and/or anti-diabetic compositions and new therapeutic methods with less or no side effects and lower toxicity for treating or preventing obesity or diabetes or IGT, and conditions associated therewith.

The present invention relates to combinations comprising a DPP IV inhibitor or a pharmaceutically acceptable salt thereof and an anti-obesity agent, or an appetite regulating agent, or a pharmaceutically acceptable salt thereof.

Preferably the present invention relates to a combination, such as a combined preparation or pharmaceutical composition, respectively, comprising a DPP IV inhibitor or a pharmaceutically acceptable salt thereof, and at least one therapeutic agent selected from the group consisting of

i) an anti-obesity agent or a pharmaceutically acceptable salt thereof,

ii) an appetite regulating agent or a pharmaceutically acceptable salt thereof, and at least one additional pharmaceutically acceptable carrier.

Preferably the combination is a pharmaceutical composition or a combined pharmaceutical preparation.

In this pharmaceutical composition, the combination partners (i) and (ii) can be administered together, one after the other or separately in one combined unit dosage form or in two sepa-rate unit dosage forms. The unit dosage form may also be a fixed combination.

The term “at least one therapeutic agent” shall mean that in addition to the DPP IV inhibitor one or more, for example two, furthermore three, active ingredients as specified according to the present invention can be combined.

The term “DPP-IV” as used herein is intended to mean dipeptidyl peptidase IV, also known as CD26. DPP-IV, a serine protease belonging to the group of post-proline/alanine cleaving amino-dipeptidases, specifically removes the two N-terminal amino acids from proteins having proline or alanine in position 2. DPP-IV can be used in the control of glucose metabolism because its substrates include the insulinotropic hormones glucagon like peptide-1 (GLP-1) and gastric inhibitory peptide (GIP). GLP-1 and GIP are active only in their intact forms; removal of their two N-terminal amino acids inactivates them.

In vivo administration of synthetic inhibitors of DPP-IV prevents N-terminal degradation of GLP-1 and GIP, resulting in higher plasma concentrations of these hormones, increased insulin secretion and, therefore, improved glucose tolerance.

The term “DPP-IV inhibitor” is intended to indicate a molecule that exhibits inhibition of the enzymatic activity of DPP-IV and functionally related enzymes, such as from 1-100% inhibition, and specially preserves the action of substrate molecules, including but not limited to GLP-1, GIP, peptide histidine methionine, substance P, neuropeptide Y, and other molecules typically containing alanine or proline residues in the second amino terminal position. Treatment with DPP-IV inhibitors prolongs the duration of action of peptide substrates and increases levels of their intact, undegraded forms leading to a spectrum of biological activities relevant to the disclosed invention.

For that purpose, chemical compounds are tested for their ability to inhibit the enzyme activity of purified CD26/DPP-IV. Briefly, the activity of CD26/DPP-IV is measured in vitro by its ability to cleave the synthetic substrate Gly-Pro-p-nitroanilide (Gly-Pro-pNA). Cleavage of Gly-Pro-pNA by DPP-IV liberates the product p-nitroanilide (pNA), whose rate of appearance is directly proportional to the enzyme activity. Inhibition of the enzyme activity by specific enzyme inhibitors slows down the generation of pNA. Stronger interaction between an inhibitor and the enzyme results in a slower rate of generation of pNA. Thus, the degree of inhibition of the rate of accumulation of pNA is a direct measure of the strength of enzyme inhibition. The accumulation of pNA is measured spectrophotometrically. The inhibition constant, Ki, for each compound is determined by incubating fixed amounts of enzyme with several different concentrations of inhibitor and substrate.

In the present context “a DPP-IV inhibitor” is also intended to comprise active metabolites and prodrugs thereof, such as active metabolites and prodrugs of DPP-IV inhibitors. An active “metabolite” is an active derivative of a DPP-IV inhibitor produced when the DPP-IV inhibitor is metabolized. A “prodrug” is a compound that is either metabolized to a DPP-IV inhibitor or is metabolized to the same metabolite(s) as a DPP-IV inhibitor.

DPP-IV inhibitors are known in the art. For example, DPP-IV inhibitors are in each case generically and specifically disclosed e.g. in WO 98/19998, DE19616 486 A1, WO 00/34241, WO 95/15309, WO 01/72290, WO01/52825, WO 9310127, WO 9925719, WO 938501, WO 9946272, WO 9967278 and WO 9967279. In each case in particular in the compound claims and the final products of the working examples, the subject matter of the final products, the pharmaceutical preparations and the claims are hereby incorporated into the present application by reference to these publications.

Preferred DPP-IV inhibitors are described in the following patent applications; WO 02053548 especially compounds 1001 to 1293 and examples 1 to 124, WO 02067918 especially compounds 1000 to 1278 and 2001 to 2159, WO 02066627 especially the described examples, WO 02/068420 especially all the compounds specifically listed in the examples I to LXIII and the described corresponding analogues, even preferred compounds are 2(28), 2(88), 2(119), 2(136) described in the table reporting IC50, WO 02083128 especially examples 1 to 13, US 2003096846 especially the specifically described compounds, WO 2004/037181 especially examples 1 to 33, WO 0168603 especially compounds of examples 1 to 109, EP1258480 especially compounds of examples 1 to 60, WO 0181337 especially examples 1 to 118, WO 02083109 especially examples 1A to 1D, WO 030003250 especially compounds of examples 1 to 166, most preferably 1 to 8, WO 03035067 especially the compounds described in the examples, WO 03/035057 especially the compounds described in the examples, US2003216450 especially examples 1 to 450, WO 99/46272 especially compounds of claims 12, 14, 15 and 17, WO 0197808 especially compounds of claim 2, WO 03002553 especially compounds of examples 1 to 33, WO 01/34594 especially the compounds described in the examples 1 to 4, WO 02051836 especially examples 1 to 712, EP1245568 especially examples 1 to 7, EP1258476 especially examples 1 to 32, US 2003087950 especially the described examples, WO 02/076450 especially examples 1 to 128, WO 03000180 especially examples 1 to 162, WO 03000181 especially examples 1 to 66, WO 03004498 especially examples 1 to 33, WO 0302942 especially examples 1 to 68, U.S. Pat. No. 6,482,844 especially the described examples, WO 0155105 especially the compounds listed in the examples 1 and 2, WO 0202560 especially examples 1 to 166, WO 03004496 especially examples 1 to 103, WO 03/024965 especially examples 1 to 54, WO 0303727 especially examples 1 to 209, WO 0368757 especially examples 1 to 88, WO 03074500 especially examples 1 to 72, examples 4.1 to 4.23, examples 5.1 to 5.10, examples 6.1 to 6.30, examples 7.1 to 7.23, examples 8.1 to 8.10, examples 9.1 to 9.30, WO 02038541 especially examples 1 to 53, WO 02062764 especially examples 1 to 293, preferably the compound of example 95 (2-{{3-(Aminomethyl)-4-butoxy-2-neopentyl-1-oxo-1,2 dihydro-6-isoquinolinyl}oxy}acetamide hydrochloride), WO 02308090 especially examples 1-1 to 1-109, examples 2-1 to 2-9, example 3, examples 4-1 to 4-19, examples 5-1 to 5-39, examples 6-1 to 6-4, examples 7-1 to 7-10, examples 8-1 to 8-8, examples 7-1 to 7-7 of page 90, examples 8-1 to 8-59 of pages 91 to 95, examples 9-1 to 9-33, examples 10-1 to 10-20, US 2003225102 especially compounds 1 to 115, compounds of examples 1 to 121, preferably compounds a) to z), aa) to az), ba) to bz), ca) to cz) and da) to dk), WO 0214271 especially examples 1 to 320 and US 2003096857, WO 20041052850 especially the specifically described compounds such as examples 1 to 42 and compounds of claim 1, DE 102 56 264 A1 especially the described compounds such as examples 1 to 181 and the compounds of claim 5, WO 04/076433 especially the compounds specifically described, such as listed in table A, preferably the compounds listed in table B, preferably compounds I to XXXXVII, or compounds of claims 6 to 49, WO 04/071454 especially the specifically described compounds e.g. compounds 1 to 53 or compounds of tables 1a to If, or compounds of claims 2 to 55, WO 02/068420 especially the compounds specifically described, such as the compounds I to LXIII or Beispiele I and analogues 1 to 140 or Beispiele 2 and analogues 1 to 174 or Beispiele 3 and analogues 1, or Beispiele 4 to 5, or Beispiele 6 and analogues 1 to 5, or Beispiele 7 and analogues 1-3, or Beispiele 8 and analogue 1, or Beispiele 9, or Beispiele 10 and analogues 1 to 531 even preferred are compounds of claim 13, WO 03/000250 especially the compounds specifically described, such as the compounds 1 to 166, preferably compounds of examples 1 to 9, WO 03/024942 especially the compounds specifically described, such compounds 1 to 59, compounds of table 1 (1 to 68), compounds of claims 6, 7, 8, 9, WO 03024965024942 especially the compounds specifically described, such compounds 1 to 54, Wo03002593 especially the compounds specifically described, such compounds table 1 or of claims 2 to 15, WO03037327 especially the compounds specifically described, such compounds of examples 1 to 209 WO 03/000250 especially the compounds specifically described, such as the compounds 1 to 166, preferably compounds of examples 1 to 9, WO 03/024942 especially the compounds specifically described, such compounds 1 to 59, compounds of table 1 (1 to 68), compounds of claims 6, 7, 8, 9, WO 03024965024942 especially the compounds specifically described, such compounds 1 to 54, Wo03002593 especially the compounds specifically described, such compounds table 1 or of claims 2 to 15, WO03037327 especially the compounds specifically described, such compounds of examples 1 to 209, WO0238541, WO0230890, WO 03/000250 especially the compounds specifically described, such as the compounds 1 to 166, preferably compounds of examples 1 to 9, WO 03/024942 especially the compounds specifically described, such compounds 1 to 59, compounds of table 1 (1 to 68), compounds of claims 6, 7, 8, 9, WO 03024965 especially the compounds specifically described, such compounds 4 to 54, Wo03002593 especially the compounds specifically described, such compounds table 1 or of claims 2 to 15, WO03037327 especially the compounds specifically described, such compounds of examples 1 to 209, WO0238541 especially the compounds specifically described, such compounds of examples 1 to 53, WO 03/002531 especially the compounds specifically described preferably the compounds listed on page 9 to 13, most preferably the compounds of examples 1 to 46 and even preferred compound of example 9, U.S. Pat. No. 6,395,767 preferably compound of examples 1 to 109 most preferably compound of example 60.

Published patent application WO 9819998 discloses N—(N′-substituted glycyl)-2-cyano pyrrolidines, in particular 1-[2-[5-Cyanopyridin-2-yl]amino]-ethylamino]acetyl-2-cyano-(S) pyrrolidine (NVP-DPP728).

DE19616 486 A1 discloses val-pyr, val-thiazolidide, isoleucyl-thiazolidide, isoleucyl-pyrrolidide, and fumar salts of isoleucyl-thiazolidide and isoleucyl-pyrrolidide.

Published patent application WO 0034241 and published U.S. Pat. No. 6,110,949 disclose N-substituted adamantyl-amino-acetyl-2-cyano pyrrolidines and N-(substituted glycyl)-4-cyano pyrrolidines respectively. DPP-IV inhibitors of interest are specially those cited in claims 1 to 4. In particular these applications describe the compound 1-[[(3-Hydroxy-1-adamantyl) amino]acetyl]-2-cyano-(S)-pyrrolidine (also known as LAF237).

Published patent application WO 9515309 discloses amino acid 2-cyanopyrrolidine amides as inhibitors of DPP-IV Published patent application WO 9529691 discloses peptidyl derivates of diesters of alpha-aminoalkylphosphonic acids, particularly those with proline or related structures. DPP-IV inhibitors of interest are specially those cited in Table 1 to 8.

In WO 01/72290 DPP-IV inhibitors of interest are specially those cited in example 1 and claims 1, 4, and 6.

WO01/52825 specially discloses (S)-1-{2-[5-cyanopyridin-2yl)amino]ethyl-aminoacetyl)-2-cyano-pyrrolidine or (S)-1-[(3-hydroxy-1-adamantyl)amino]acetyl-2-cyano-pyrrolidine.

Published patent application WO 9310127 discloses proline boronic esters useful as DPP-IV inhibitors. DPP-IV inhibitors of interest are specially those cited in examples 1 to 19.

Published patent application WO 9925719 discloses sulphostin, a DPP-IV inhibitor prepared by culturing a Streptomyces microorganism.

Published patent application WO 9938501 discloses N-substituted 4-8 membered heterocyclic rings. DPP-IV inhibitors of interest are specially those cited in claims 15 to 20.

Published patent application WO 9946272 discloses phosphoric compounds as inhibitors of DPP-IV. DPP-IV inhibitors of interest are specially those cited in claims 1 to 23.

Published patent applications WO 9967278 and WO 9967279 disclose DPP-IV prodrugs and inhibitors of the form A-B-C where C is either a stable or unstable inhibitor of DPP-IV.

Any of the substances disclosed in the above mentioned patent documents, hereby included by reference, are considered potentially useful as DPP-IV inhibitors to be used in carrying out the present invention.

In a further preferred embodiment, the DPP-IV inhibitor is a N-peptidyl-O-aroyl hydroxylamine or a pharmaceutically acceptable salt thereof. Aroyl is, for example, naphthylcarbonyl; or benzoyl which is unsubstituted or mono- or disubstituted, for example, by lower alkoxy, lower alkyl, halogen or, preferably, nitro. The peptidyl moiety comprises preferably two α-amino acids, e.g. glycine, alanine, leucine, phenylalanine, lysine or proline, of which the one attached directly to the hydroxylamine nitrogen atom is preferably proline.

Preferably, the N-peptidyl-O-aroyl hydroxylamine is a compound of formula VII 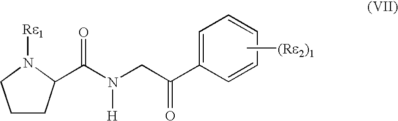
wherein
j is 0, 1 or 2;
Rε₁ represents the side chain of a natural amino acid; and
Rε₂ represents lower alkoxy, lower alkyl, halogen or nitro;
or a pharmaceutically acceptable salt thereof.

In a very preferred embodiment of the invention, the N-peptidyl-O-aroyl hydroxylamine is a compound of formula VIIa 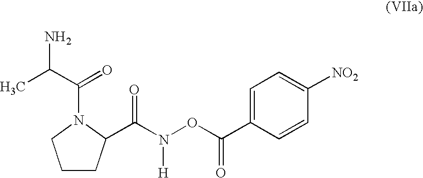
or a pharmaceutically acceptable salt thereof.

N-Peptidyl-O-aroyl hydroxylamines, e.g. of formula VII or VIIa, and their preparation are described by H. U. Demuth et al. in J. Enzyme Inhibition 1988, Vol. 2, pages 129-142, especially on pages 130-132.

Preferred DPP-IV inhibitors are N-substituted adamantyl-amino-acetyl-2-cyano pyrrolidines, N (substituted glycyl)-4-cyano pyrrolidines, N—(N′-substituted glycyl)-2-cyanopyrrolidines, N-aminoacyl thiazolidines, N-aminoacyl pyrrolidines, L-allo-isoleucyl thiazolidine, L-threo-isoleucyl pyrrolidine, and L-allo-isoleucyl pyrrolidine, 1-[2-[(5-cyanopyridin-2-yl) amino]ethylamino]acetyl-2-cyano-(S)-pyrrolidine and pharmaceutical salts thereof.

Preferred DPP-IV inhibitors are those described by Mona Patel and col. (Expert Opinion Investig Drugs. 2003 April; 12(4):623-33) on the paragraph 5, especially P32/98, K-364, FE-999011, BDPX, NVP-DDP-728 and others, which publication is hereby incorporated by reference especially the described DPP-IV inhibitors.

FE-999011 is described in the patent application WO 95/15309 page 14, as compound No. 18.

Another preferred inhibitor is the compound BMS-477118 disclosed in U.S. Pat. No. 6,395,767 (compound of example 60) also known as is (1S,3S,5S)-2-[(2S)-2-amino-2-(3-hydroxytricyclo[3.3.1.1^{3,7}]dec-1-yl)-1-oxoethyl]-2-azabicyclo[3.1.0]hexane-3-carbonitrile, benzoate (1:1) as depicted in Formula M of the patent application WO 2004/052850 on page 2, and the corresponding free base, (1S,3S,5S)-2-[(2S)-2-amino-2-(3-hydroxy-tricyclo[3.3.1.1^{3,7}]dec-1-yl)-1-oxoethyl]-2-azabicyclo[3.1.0]hexane-3-carbonitrile (M′) and its monohydrate (M″) as depicted in Formula M of the patent application WO 20041052850 on page 3.

Another preferred inhibitor is the compound GSK23A disclosed in WO 03/002531 (example 9) also known as (2S,4S)-1-((2R)-2-Amino-3-[(4-methoxybenzyl)sulfonyl]-3-methylbutanoyl)-4-fluoropyrrolidine-2-carbonitrile hydrochloride.

Other very preferred DPP-IV inhibitors of the invention are described in the International patent application WO 02/076450 (especially the examples 1 to 128) and by Wallace T. Ashton (Bioorganic & Medicinal Chemistry Letters 14 (2004) 859-863) especially the compound 1 and the compounds listed in the tables 1 and 2. The preferred compound is the compound 21e (table 1) of formula

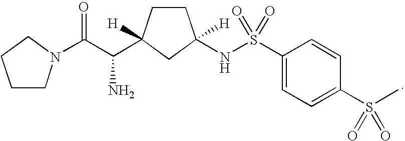

P32/98 or P3298 (CAS number: 251572-86-8) also known as 3-[(2S,3S)-2-amino-3-methyl-1-oxopentyl]thiazolidine can be used as 3-[(2S,3S)-2-amino-3-methyl-1-oxopentyl]thiazolidine and (2E)-2-butenedioate (2:1) mixture such as shown below 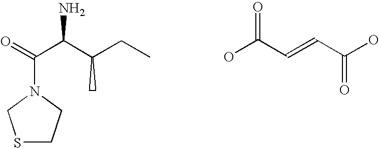
and is described in WO 99/61431 in the name of Probiodrug and also the compound P 93/01.

Other preferred DPP-IV inhibitors are the compounds disclosed in the patent application WO 02/083128 such as in the claims 1 to 5. Most preferred DPP-IV inhibitors are the compounds specifically described by the examples 1 to 13 and the claims 6 to 10.

Other preferred DPP-IV inhibitors are described in the patent applications WO 2004/037169 especially those described in the examples 1 to 48 and WO 02/062764 especially the described examples 1 to 293, even preferred are the compounds 3-(aminomethyl)-2-isobuthyl-1-oxo-4-phenyl-1,2-dihydro-6-isoquinolinecarboxamide and 2-{[3-(aminomethyl)-2-isobuthyl-4-phenyl-1-oxo-1,2-dihydro-6-isoquinolyl]oxy}acetamide described on page 7 and also in the patent application WO2004/024184 especially in the reference examples 1 to 4.

Other preferred DPP-IV inhibitors are described in the patent application WO 03/004498 especially examples 1 to 33 and most preferably the compound of the formula 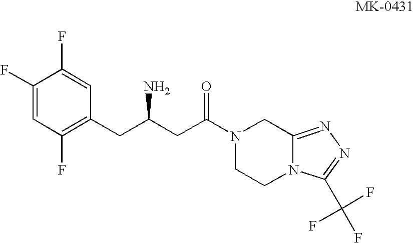
described by the example 7 and also known as MK-0431.

Preferred DPP-IV inhibitors are also described in the patent application WO 2004/037181 especially examples 1 to 33, most preferably the compounds described in the claims 3 to 5.

Preferred DPP-IV inhibitors are N-substituted adamantyl-amino-acetyl-2-cyano pyrrolidines, N (substituted glycyl)-4-cyano pyrrolidines, N—(N′-substituted glycyl)-2-cyanopyrrolidines, N-aminoacyl thiazolidines, N-aminoacyl pyrrolidines, L-allo-isoleucyl thiazolidine, L-threo-isoleucyl pyrrolidine, and L-allo-isoleucyl pyrrolidine, 1-[2-[(5-cyanopyridin-2-yl) amino]ethylamino]acetyl-2-cyano-(S)pyrrolidine and pharmaceutical salts thereof.

Especially preferred are 1-{2-[(5-cyanopyridin-2-yl) amino]ethylamino}acetyl-2 (S)-cyano-pyrrolidine dihydrochloride (DPP728), of formula 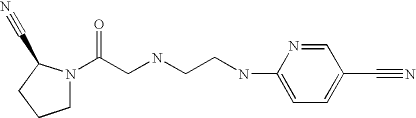
especially the dihydrochloride thereof,
and (S)-1-[(3-hydroxy-1-adamantyl)amino]acetyl-2-cyano-pyrrolidine (LAF237) of formula 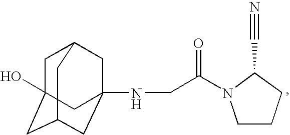
and L-threo-isoleucyl thiazolidine (compound code according to Probiodrug: P32/98 as described above), MK-0431, GSK23A, BMS-477118, 3-(aminomethyl)-2-isobuthyl-1-oxo-4-phenyl-1,2-dihydro-6-isoquinolinecarboxamide and 2-{[3-(aminomethyl)-2-isobuthyl-4-phenyl-1-oxo-1,2-dihydro-6-isoquinolyl]oxy}acetamide and optionally in any case pharmaceutical salts thereof.

DPP728 and LAF237 are the very preferred compounds and are specifically disclosed in Example 3 of WO 98/19998 and Example 1 of WO 00/34241, respectively. The DPP-IV inhibitor P32/98 (see above) is specifically described in Diabetes 1998, 47, 1253-1258. DPP728 and LAF237 can be formulated as described on page 20 of WO 98/19998 or in WO 00/34241. The preferred formulations for the administration of LAF237 are described in the U.S. provisional application No. 60/604,274.

Especially preferred are orally active DPP-IV inhibitors. In a further embodiment, preferred DPP-IV inhibitors are preferably not dipeptidic compounds and derivatives.

Anti-obesity agents or appetite regulating agents are described below.

Such agents may be selected from the group consisting of CART (cocaine amphetamine regulated transcript) agonists, catecholaminergic agents (e.g. diethylpropion, phentermine, phenylpropanolamine, mazindol), NPY (neuropeptide Y) antagonists, MC 4 (melanocortin 4) agonists, MC 3 (melanocortin 3) agonists, orexin antagonists, TNF (tumor necrosis factor) agonists, CRF (corticotropin releasing factor) agonists, CRF BP (corticotropin releasing factor binding protein) antagonists, urocortin agonists, a melanin concentrating hormone antagonists, β3 adrenergic receptor agonists, MSH (melanocyte-stimulating hormone) agonists or mimetics, MCH (melanocyte-concentrating hormone) antagonists, thyromimetic agents, dehydroepiandrosterone or an analog thereof, glucocorticoid receptor agonist or antagonist, ciliary neurotrophic factors, human agouti-related protein antagonists, CCK (cholecystokinin) agonists, monoamine re-uptake inhibitors, serotonin re-uptake inhibitors, serotonin and noradrenaline re-uptake inhibitors, mixed serotonin and noradrenergic compounds, 5HT (serotonin) agonists, dopamine agonists, bombesin agonists, galanin antagonists, growth hormone, growth factors such as prolactin or placental lactogen, growth hormone releasing compounds, TRH (thyreotropin releasing hormone) agonists, UCP 2 or 3 (uncoupling protein 2 or 3) modulators, leptin agonists, DA agonists (bromocriptin, doprexin), lipase/amylase inhibitors, RXR (retinoid X receptor) modulators, TR β agonists, AGRP (Agouti related protein) inhibitors, opioid antagonists (such as naltrexone), exendin-4, PACAP (pituitary adenylyl cyclase activationg peptide), cannabinoid receptor antagonists, GLP-1 and ciliary neurotrophic factor.

The dosage of the anti-obesity agent or appetite regulating agent administered will also be generally dependent upon the health of the subject being treated, the extent of obesity treatment desired, the nature and kind of concurrent therapy, if any, and the frequency of treatment and nature of the effect desired. In general, the dosage of the anti-obesity agent is generally in the range of from about 0.001 to about 50 mg/kg body weight of the subject per day, preferably from about 0.1 to about 10 mg/kg body weight of the subject per day, administered as a single or divided dose. However, some variability in the general dosage range may also be required depending upon the age, weight, and species of the patient, the intended route of administration, and the progress and degree of severity of the obesity being treated.

Preferred examples of β3-adrenergic receptor agonists are selected from the group consisting of {4-[2-(2-[6-aminopyridin-3-yl]-2(R)-hydroxyethylamino)ethoxy]phenyl}acetic acid, {4-[2-(2-[6-aminopyridin-3-yl]-2(R)-hydroxyethylamino)ethoxy]phenyl}benzoic acid, {4-[2-(2-[6-aminopyridin-3-yl]-2(R)-hydroxyethylamino)ethoxy]phenyl}propionic acid, and {4-[2-(2-[6-aminopyridin-3-yl]-2(R)-hydroxyethylamino)ethoxy]phenoxy}acetic acid.

In a first embodiment of the invention the appetite regulating agent (amphetamine-related appetite suppressant) is phentermine or phentermine hydrochloride. Phentermine may be prepared as described in U.S. Patent No. 2,408,345, the disclosure of which is incorporated herein by reference. When the anti-obesity agent is phentermine, the dosage of phentermine is from about 0.01 to about 10 mg/kg body weight of the subject per day, preferably from about 0.1 to about 1 mg/kg body weight of the subject per day. Phentermine is preferably administered from 15 to 100 mg per day, preferably 30 to 50 mg per day most preferably 37.5 mg per day and optionally in divided doses two to three times per day.

Another appetite regulating agent is the gut hormone peptide YY (PYY) (Batterham R L, Bloom S R “The gut hormone peptide YY regulates appetite”—Ann N Y Acad. Sci. (2003 Jun); 994:162-8). Preferred is gut hormone fragment peptide YY3-36 peptide (YY₃₋₃₆).

In one embodiment of the invention the anti-obesity agent is leptin.

In another embodiment the anti-obesity agent is dexamphetamine or amphetamine.

In another embodiment the anti-obesity agent (serotonin agonist) is fenfluramine or dexfenfluramine or dexfenfluramine hydrochloride. The particularly preferred serotoninergic agents fenfluramine and dexfenfluramine may be prepared as described in U.S. Pat. No. 3,198,834, the disclosure of which is incorporated herein by reference. When the anti-obesity agent is fenfluramine or dexfenfluramine, the dosage range of fenfluramine or dexfenfluramine is from about 0.01 to about 30 mg/kg body weight of the subject per day, preferably from about 0.1 to about 1 mg/kg body weight of the subject per day. Fenfluramine is preferably administered from 20 to 120 mg per day, preferably 40 to 80 mg per day most preferably 60 mg per day and optionally in divided doses two to three times per day. Dexfenfluramine or dexfenfluramine hydrochloride are preferably administered from 10 to 120 mg per day, preferably 20 to 60 mg per day most preferably 30 mg per day and optionally in divided doses two to three times per day e.g. 2 times 15 mg per day

In still another embodiment the anti-obesity agent (serotonin and noradrenaline reuptake inhibitor) is sibutramine or its hydrochloride salt. The particularly preferred monoamine reuptake inhibitor sibutramine may be prepared as described in U.S. Pat. No. 4,929,629, the disclosure of which is incorporated herein by reference. VVhen the anti-obesity agent is sibutramine, the dosage of sibutramine is from about 0.01 to about 30 mg/kg body weight of the subject per day, preferably from about 0.1 to about 1 mg/kg body weight of the subject per day.

Sibutramine or its hydrochloride salt is preferably administered from 2 to 60 mg per day, preferably 5 to 25 mg or 10 to 20 mg per day most preferably 15 mg per day and optionally in divided doses two to three times per day. Preferably sibutramine is used in the form of Meridia®.

The particularly preferred dopamine agonist bromocriptine may be prepared as described in U.S. Pat. Nos. 3,752,814 and 3,752,888, the disclosures of which are incorporated herein by reference. When the anti-obesity agent is bromocriptine, the dosage range of bromocriptine is from about 0.01 to about 10 mg/kg body weight of the subject per day, preferably from about 0.1 to about 10 mg/kg body weight of the subject per day.

In a further embodiment the anti-obesity agent (lipase inhibitor) is dexfenfluramine hydrochloride or orlistat. Orlistat is a known compound useful for the control or prevention of obesity and hyperlipidemia. See, U.S. Pat. No. 4,598,089, issued Jul. 1, 1986, which also discloses processes for making orlistat and U.S. Pat. No. 6,004,996, which discloses appropriate pharmaceutical compositions. Orlistat is preferably orally administered from 60 to 720 mg per day in divided doses two to three times per day. Preferred is wherein from 180 to 360 mg, most preferably 360 mg per day of a lipase inhibitor is administered to a subject, preferably in divided doses two or, particularly, three times per day e.g. 3 times 120 mg per day. Orlistat is being marketed under the trade name Xenical®. Preferably orlisat is used in the form of Xenical®.

In another embodiment the anti-obesity agent mazindol or phentermine. Mazindol is preferably administered from 0.5 to 5 mg per day, preferably 1 mg per day and optionally in divided doses two to three times per day. Phentermine is preferably administered from 10 to 50 mg per day, preferably 15 to 37.5 mg per day most preferably 30 mg per day and optionally in divided doses two to three times per day. Preferably phentermine is used in the form of Ionamin®.

In a preferred embodiment, the antiobesity agent is phen-fen, which is the combination of fenfluramine or its hydrochloride and phentermin.

In still another embodiment the anti-obesity agent is phendimetrazine or its tartrate salt, diethylpropion or its hydrochloride salt, fluoxetine, sertaline or its hydrochloride salt, ephedrine or its sulphate salt, bupropion, topiramate, benzphetamine or its hydrochloride salt, phenylpropanolamine or its hydrochloride salt, or ecopipam. Fluoxetine or diethylpropion are preferably administered from 20 to 120 mg per day, preferably 40 to 80 mg per day, most preferably 60 mg (fluoxetine) or 75 mg (diethylpropion) per day and optionally in divided doses two to three times per day. Diethylpropion is preferably taken 3 times daily (3×25 mg). Preferably Diethylpropion is used in the form of Tenuate®.

Preferred are combinations, such as combined preparations or pharmaceutical compositions, respectively, comprising the DPP-IV inhibitor of formula (I) or a pharmaceutically accepted salt thereof and as second active agent an active agent selected from the group consisting of phentermine, leptin, bromocriptine, dexamphetamine, amphetamine, fenfluramine, dexfenfluramine, sibutramine, orlistat, dexfenfluramine, mazindol, phentermine, phendimetrazine, diethylpropion, fluoxetine, bupropion, topiramate, diethylpropion, benzphetamine, phenylpropanolamine or ecopipam, ephedrine, pseudoephedrine and pharmaceutical salts thereof.

Furthermore preferred are combinations, such as a combined preparations or pharmaceutical compositions, respectively, comprising the DPP-IV inhibitor of formula (I) or a pharmaceutically accepted salt thereof and one active agent selected from the group consisting of orlistat, sibutramine, diethylpropion, phen-fen and phentermine.

The corresponding active ingredients or a pharmaceutically acceptable salt thereof may also be used in form of a solvate, such as a hydrate or including other solvents, used for crystallization.

The compounds to be combined can be present as pharmaceutically acceptable salts. If these compounds have, for example, at least one basic center, they can form acid addition salts. Corresponding acid addition salts can also be formed having, if desired, an additionally present basic center. The compounds having an acid group (for example COOH) can also form salts with bases.

All of these marketed products may be utilized in as such for combination therapy according to the present invention.

The structure of the active agents identified by generic or tradenames may be taken from the actual edition of the standard compendium “The Merck Index” or from databases, e.g. Patents International (e.g. IMS World Publications). The corresponding content thereof is hereby incorporated by reference. Any person skilled in the art is fully enabled to identify the active agents and, based on these references, likewise enabled to manufacture and test the pharmaceutical indications and properties in standard test models, both in vitro and in vivo.

All the more surprising is the experimental finding that the combined administration of a DPP IV inhibitor or a salt thereof and at least one therapeutic agent selected from the group consisting of (i) to (ii) results not only in a beneficial, especially a synergistic, therapeutic effect, but also in additional benefits resulting from the combined treatment and further surprising beneficial effects compared to a monotherapy applying only one of the pharmaceutically active compounds used in the combinations disclosed herein.

It can be shown by established test models and especially those test models described herein that the combination of the DPP-IV inhibitor of formula (I) with at least one therapeutic agent selected from the group consisting of (i) to (ii) results in a more effective prevention or preferably treatment of diseases specified in the following. In particular, it can be shown by established test models and especially those test models described herein that the combination of the present invention results in a more effective prevention or preferably treatment of diseases specified hereinafter.

If taken simultaneously, this results not only in a further enhanced beneficial, especially a synergistic, therapeutic effect, but also in additional benefits resulting from the simultaneous treatment such as a surprising prolongation of efficacy, a broader variety of therapeutic treatment and surprising beneficial effects on diseases and conditions associated with diabetes mellitus IGT or obesity, for a number of combinations as described herein.

The term “potentiation” shall mean an increase of a corresponding pharmacological activity or therapeutical effect, respectively. Potentiation of one component of the combination according to the present invention by co-administration of another component according to the present invention means that an effect is being achieved that is greater than that achieved with one component alone.

The term “synergistic” shall mean that the drugs, when taken together, produce a total joint effect that is greater than the sum of the effects of each drug when taken alone.

Moreover, for a human patient, especially for elderly people, it is more convenient and easier to remember to take two tablets at the same time, e.g. before a meal, than staggered in time, i.e. according to a more complicated treatment schedule. More preferably, both active ingredients are administered as a fixed combination, i.e. as a single tablet, in all cases described herein. Taking a single tablet is even easier to handle than taking two tablets at the same time. Furthermore, the packaging can be accomplished with less effort.

The person skilled in the pertinent art is fully enabled to select a relevant and standard animal test model to prove the hereinbefore and hereinafter indicated therapeutic indications and beneficial effects.

The pharmaceutical activities as effected by administration of the combination of the active agents used according to the present invention can be demonstrated e.g. by using corresponding pharmacological models known in the pertinent art.

The evaluation of the cardiovascular benefic effects especially in diabetes of the agents given alone or in combination can be performed using models such as the Zucker fatty rat as described in the publication of Nawano et al., Metabolism 48: 1248-1255, 1999. Also, studies using diabetic spontaneously hypertensive rats are described in the publication of Sato et al., Metabolism 45:457-462, 1996.

To evaluate the antihypertensive activity of the combination according to the invention, for example, the methodology as described by Lovenberg W: Animal models for hypertension research. Prog. Clin. Biol. Res. 1987, 229, 225-240 may be applied. For the evaluation that the combination according to the present invention may be used for the treatment of congestive heart failure, for example, the methods as disclosed by Smith H J, Nuttall A: Experimental models of heart failure. Cardiovasc Res 1985, 19, 181-186 may be applied. Molecular approaches such as transgenic methods are also described, for example by Luft et al.: Hypertension-induced end-organ damage. “A new transgemic approach for an old problem”—Hypertension 1999, 33, 212-218.

To evaluate the anti-obesity activity i.e. weight loss, reduction of the plasma triacylglycerol levels, fat excretion in feces of mice, body, liver and parametrial adipose tissue weights reduction, energy intake, and liver triacylglycerol and total cholesterol concentrations reduction, of the combination according to the invention, for example, the methodology as described by Han L K (J Nutr. 2002 August; 132(8):2241-5.) may be applied.

For the evaluation that the combination according to the present invention may be used for the treatment of congestive heart failure, for example, the methods as disclosed by Smith H J, Nuftall A: Experimental models of heart failure. Cardiovasc Res 1985, 19, 181-186 may be applied. Also, rat models of hypertension and cardiac failure as described by Doggrell S A and Brown L (Cardiovasc Res 1998, 39: 89-105) may be used for the pharmacological evaluation of the combination. Also, rat models as described in the prior art may be used for the pharmacological evaluation of the combination. Molecular approaches such as transgenic methods are also described, in the prior art.

The insulin secretion enhancing properties of the combination according to the present invention may be determined by following the methodology as disclosed, for example, in the publication of T. Ikenoue et al. Biol. Phamn. Bull. 29(4), 354-359 (1997).

The simultaneous evaluation of the less increase of weight action the cardiovascular action and of the glucose utilization effects of the agents given alone or in combination can be performed using models such as the Zucker fatty rat as described in the publication of Nawano et al., Metabolism 48: 1248-1255, 1999. Also, studies using diabetic spontaneously hypertensive rats are described in the publication of Sato et al., Metabolism 45:457-462, 1996. Furthermore, rat models such as the Cohen-Rosenthal diabetic hypertensive rat (Rosenthal et al., Hypertension. 1997; 29:1260-1264) may also be used for the simultaneous assessments of the effects of the combination on blood pressure, increase of weight and glucose metabolism.

The corresponding subject matter of these references is herewith incorporated by reference in this specification.

Accordingly, the combination according to the present invention may be user, e.g., for the prevention, delay of progression or treatment of diseases and disorders that may be inhibited by DPP IV inhibition, that may be inhibited by the enhancement of insulin secretion and that may be inhibited by insulin sensitization.

Especially, the combination according to the present invention may be used, e.g., for the prevention, delay of progression or treatment of diseases and disorders selected from the group consisting of hypertension (including but not limited to isolated systolic hypertension and familial dyslipidemic hypertension), congestive heart failure, left ventricular hypertrophy, peripheral arterial disease, diabetes, especially type 2 diabetes mellitus, diabetic retinopathy, macular degeneration, cataract, diabetic nephropathy, glomerulosclerosis, chronic renal failure, diabetic neuropathy, syndrome X, premenstrual syndrome, coronary heart disease, angina pectoris, thrombosis, atherosclerosis, myocardial infarction, transient ischemic attacks, stroke, vascular restenosis, hyperglycemia, hyperinsulinemia, hyperlipidemia, hypertryglyceridemia, insulin resistance, impaired glucose metabolism, conditions of impaired glucose tolerance (IGT), conditions of impaired fasting plasma glucose, obesity, conditions associated with obesity, erectile dysfunction, skin and connective tissue disorders, foot ulcerations and ulcerative colitis, endothelial dysfunction and impaired vascular compliance. Preferably, said combination may be used for the treatment of hypertension, especially isolated systolic hypertension (ISH), congestive heart failure, endothelial dysfunction, impaired vascular compliance, impaired glucose tolerance and type II diabetes mellitus.

A “disease or condition which may be inhibited by a DPP-IV inhibitor” as defined in this application comprises, but is not limited to insulin resistance, impaired glucose metabolism, conditions of impaired glucose tolerance, conditions of impaired fasting plasma glucose, obesity, diabetic retinopathy, macular degeneration, cataracts, diabetic nephropathy, glomerulosclerosis, diabetic neuropathy, erectile dysfunction, premenstrual syndrome, coronary heart disease, hypertension, angina pectoris, myocardial infarction, stroke, vascular restenosis, skin and connective tissue disorders, foot ulcerations and ulcerative colitis, endothelial dysfunction and impaired vascular compliance. Preferably, a “disease or condition which may be inhibited by a DPP-IV inhibitor” is selected from impaired glucose metabolism, conditions of impaired glucose tolerance, conditions of impaired fasting plasma glucose, obesity, diabetic retinopathy, diabetic nephropathy, diabetic neuropathy and foot ulcerations.

It has surprisingly been found that the combination of a DPP-IV inhibitor and a anti-obesity agent, as described in the present invention, leads to a decrease of ISH (most common for of hypertension in people over 50 years) and pulse rate, both in hypertensive patients having type 2 diabetes mellitus and in hypertensive patients that do not have type 2 diabetes mellitus.

In the present description, the term “treatment” includes both prophylactic or preventative treatment as well as curative or disease suppressive treatment, including treatment of patients at risk of contracting the disease or suspected to have contracted the disease or disorder as well as ill patients. This term further includes the treatment for the delay of progression of the disease.

The term “curative” as used herein means efficacy in treating ongoing diseases, disorder or conditions.

The term “prophylactic” means the prevention of the onset or recurrence of diseases, disorders or conditions to be treated.

The term “delay of progression” as used herein means administration of the combination to patients being in a pre-stage or in an early phase of the disease to be treated, in which patients for example a pre-form of the corresponding disease is diagnosed or which patients are in a condition, e.g. during a medical treatment or a condition resulting from an accident, under which it is likely that a corresponding disease will develop.

Preferably, the jointly therapeutically effective amounts of the active agents according to the combination of the present invention can be administered simultaneously or sequentially in any order, e.g. separately or in a fixed combination.

Under certain circumstances, drugs with different mechanisms of action may be combined. However, just considering any combination of drugs having different modes of action but acting in the similar field does not necessarily lead to combinations with advantageous effects.

All the more surprising is the experimental finding that the combined administration of a DPP-IV inhibitor according to the present invention, or, in each case, a pharmaceutically acceptable form thereof, results not only in a beneficial, especially a potentiating or a synergistic, therapeutic effect. Independent thereof, additional benefits resulting from combined treatment can be achieved such as a surprising prolongation of efficacy, a broader variety of therapeutic treatment and surprising beneficial effects on diseases and conditions associated with diabetes (e.g. less gain of weight or less cardiovascular side effects) and conditions associated with obesity (e.g. less cardiovascular side effects, improved glycaemic control and other side effects as described herein). An additional and preferred aspect of the present invention is the prevention, delay of progression or treatment of the condition of isolated systolic hypertension and impaired vascular compliance which means decreased vascular elasticity.

The diseases, disorders or conditions related to diabetes, particularly type 2 diabetes mellitus, includes but are not limited to diabetic nephropathy, diabetic retinopathy and diabetic neuropathy, macular degeneration, coronary heart disease, myocardial infarction, diabetic cardiomyopathy, myocardial cell death, coronary artery diseases, peripheral arterial disease, stroke, limb ischemia, vascular restenosis, foot ulcerations, endothelial dysfunction and/or atherosclerosis.

Obesity has major adverse effects on health. Morbidly obese individuals have as much as a twelvefold increase in mortality. Morality rates rise as obesity increases, particularly when obesity is associated with increased intra-abdominal fat. It is also apparent that the degree to which obesity affects particular organ systems is influenced by susceptibility genes that vary in the population.

The diseases, disorders or conditions related to obesity includes but are not limited to Insulin Resistance and Type 2 Diabetes Mellitus; Reproductive Disorders e.g. Male hypogonadism, polycystic ovarian syndrome, oligomenorrhea or Gynecomastia; Cardiovascular Disease including coronary disease, stroke, and congestive heart failure (CHF); Pulmonary Disease such as reduced chest wall compliance, increased work of breathing, increased minute ventilation due to increased metabolic rate, and decreased total lung capacity and functional residual capacity, obstructive sleep apnea and the “obesity hypoventilation syndrome”; Gallstones and Fasting-induced cholecystitis; Cancers especially cancer of the colon, rectum, and prostate, cancer of the gallbladder, bile ducts, breasts, endometrium, cervix, and ovaries; Bone, Joint, and Cutaneous Disease, Cushing's Syndrome, Hypothyroidism, Insulinoma, Craniopharyngioma and Other Disorders Involving the Hypothalamus. Furthermore, it has been found that the chronic co-administration of a DPP-IV inhibitor imparts the beneficial effect on blood vessel morphology and function and results in a decrease of vascular stiffness and correspondingly in a maintenance and in an improvement of vascular compliance.

Accordingly, it has been found that the addition of a DPP-IV inhibitor to that of an anti-obesity agent would potentiate the effect on systolic blood pressure and further improve vascular stiffness/compliance. The benefit of these combinations may also extend to an additional or potentiated effect on endothelial function, and improve vascular function and structure in various organs/tissues including the kidney, heart, eye and brain. Through the reduction in glucose levels, an anti-thrombotic and anti-atherosclerotic effect can also be demonstrated. Reduction of glucose would prevent or minimize the glycosylation of any structural or functional protein within the cardio-renal system. This effect proves to be highly beneficial by evoking an additive or synergistic effect on vascular function/structure when the combination is administered.

Additionally, insulin resistance may contribute, in part, to the development of diabetes, hypertension and atherosclerosis (Fukuda et al., 2001). Administration of a combination as described in the present invention will evoke further antihypertensive effects, improve vascular dynamics in hypertensive patients to a greater extent than after administration of either agent given alone. Interestingly, the administration of the combination will partially restore insulin sensitivity by preventing renin angiotensin system-induced impairment of insulin signaling pathways while at the same time raise insulin levels and improve glucose utilization. Consequently, combined administration will simultaneously improve both the metabolic and cardiovascular abnormalities, two conditions that often coexist in patients.

The results are promising since, when compared to placebo, the claimed combinations result in a greater reduction in body weight and allowed a lowering of HbA_{1c} levels, fasting glucose concentrations and triglyceride levels, despite a significant reduction in hypoglycaemic agents, in diabetic patients. Our combinations improve the glycaemic control (e.g. higher reduction in fasting plasma glucose) in obese subjects with glucose intolerance as well as in IGT patients or Type 2 diabetic patients treated by diet, oral drugs or insulin.

Further benefits are that lower doses of the individual drugs to be combined according to the present invention can be used to reduce the dosage, for example, that the dosages need not only often be smaller but are also applied less frequently, or can be used in order to diminish the incidence of side effects. This is in accordance with the desires and requirements of the patients to be treated.

For example, it has turned out that the combination according to the present invention provides benefit especially in the treatment of diabetic patients and obese patients, e.g. reducing the risk of negative cardiovascular events, reducing risk of side effects, controlling increase of weight (in diabetic patients). Our combinations reduce in particular valvular heart diseases e.g. valvular regurgitation or valvulopathy.

The DPP-IV inhibitor according to the present invention has proven to be useful in the treatment of type 2 diabetes mellitus and can likewise be used for the reduction of blood pressure in for example improving microalbuminuria. The combination according to the invention may be merely used for the treatment of diabetes, especially type 2 diabetes mellitus, IGT and obesity. In view of reduced dose of the DPP-IV inhibitor or anti-obesity agent used according to the present invention, there is a considerable safety profile of the combination making it suitable for first line therapy.

Further benefits when applying the composition of the present invention are that lower doses of the individual drugs to be combined according to the present invention can be used to reduce the dosage, for example, that the dosages need not only often be smaller but are also applied less frequently, or can be used in order to diminish the incidence of side effects. This is in accordance with the desires and requirements of the patients to be treated.

Preferably, the jointly therapeutically effective amounts of the active agents according to the combination of the present invention can be administered simultaneously or sequentially in any order, separately or in a fixed combination.

The pharmaceutical composition according to the present invention as described herein before and hereinafter may be used for simultaneous use or sequential use in any order, for separate use or as a fixed combination.

Accordingly, the invention furthermore relates to a method for the prevention of, delay of progression of, treatment of a disease or condition selected from the group consisting of

(a) type 2 diabetes mellitus and related diseases, disorders or conditions (including but not limited to diabetic nephropathy, diabetic retinopathy and diabetic neuropathy);

(b) insulin resistance and syndrome X, obesity and related diseases, disorders or conditions (including but not limited to not limited to Insulin Resistance, Type 2 Diabetes Mellitus, Reproductive Disorders, Cardiovascular Disease, Pulmonary Disease, Gallstones and Fasting-induced cholecystitis, Cancers and Cutaneous Disease), Cushing's Syndrome, Hypothyroidism, Insulinoma, Craniopharyngioma and Other Disorders Involving the Hypothalamus;

(c) hypertension including hypertension in the elderly, familial dyslipidemic hypertension and isolated systolic hypertension (ISH); increased collagen formation, fibrosis, and remodeling following hypertension (antiproliferative effect of the combination); erectile dysfunction, impaired vascular compliance, stroke; all these diseases or conditions associated with or without hypertension,

(d) congestive heart failure, left ventricular hypertrophy, survival post myocardial infarction (MI), coronary artery diseases, atherosclerosis, angina pectoris, thrombosis,

(e) renal failure, especially chronic renal failure, glomerulosclerosis, nephropathy;

(f) hypothyroidism;

(g) endothelial dysfunction with or without hypertension,

(h) hyperlipidemia, hyperlipoproteinemia, hypertryglyceridemia, and hypercholesterolemia,

(i) macular degeneration, cataract, glaucoma,

(j) skin and connective tissue disorders, and

(k) restenosis after percutaneous transluminal angioplasty, and restenosis after coronary artery bypass surgery; peripheral vascular disease; comprising administering to a warm-blooded animal, including man, in need thereof a jointly effective amount of a combination of a DPP IV inhibitor or a pharmaceutically acceptable salt thereof with at least one therapeutic agent selected from the group consisting of

i) an antiobesity agent or a pharmaceutically acceptable salt thereof,

ii) an appetite regulating agent or a pharmaceutically acceptable salt thereof; and at least one additional pharmaceutically acceptable carrier.

Furthermore, the present invention relates to a combination according to the present invention for use as a medicament.

Furthermore, the present invention relates to the use of a DPP IV inhibitor or a pharmaceutically acceptable salt thereof in combination with at least one therapeutic agent selected from the group consisting of

i) an antiobesity agent or a pharmaceutically acceptable salt thereof,

ii) an appetite regulating agent or a pharmaceutically acceptable salt thereof, for the manufacture of a medicament for the prevention of, delay of progression of, or treatment of a disease or condition selected from the group consisting of

(a) type 2 diabetes mellitus and related diseases, disorders or conditions (including but not limited to diabetic nephropathy, diabetic retinopathy and diabetic neuropathy);

(b) insulin resistance and syndrome X, obesity and related diseases, disorders or conditions (including but not limited to not limited to Insulin Resistance, Type 2 Diabetes Mellitus, Reproductive Disorders, Cardiovascular Disease, Pulmonary Disease, Gallstones and Fasting-induced cholecystitis, Cancers and Cutaneous Disease, Cushing's Syndrome, Hypothyroidism, Insulinoma, Craniopharyngioma and Other Disorders Involving the Hypothalamus);

(c) hypertension including hypertension in the elderly, familial dyslipidemic hypertension, and isolated systolic hypertension (ISH); increased collagen formation, fibrosis, and remodeling following hypertension (antiproliferative effect of the combination); erectile dysfunction, impaired vascular compliance, stroke; all these diseases or conditions associated with or without hypertension,

(d) congestive heart failure, left ventricular hypertrophy, survival post myocardial infarction (MI), coronary artery diseases, atherosclerosis, angina pectoris, thrombosis,

(e) renal failure, especially chronic renal failure, glomerulosclerosis, nephropathy;

(f) hypothyroidism;

(g) endothelial dysfunction with or without hypertension,

(h) hyperlipidemia, hyperlipoproteinemia, hypertryglyceridemia, and hypercholesterolemia,

(i) macular degeneration, cataract, glaucoma,

(j) skin and connective tissue disorders, and

(k) restenosis after percutaneous transluminal angioplasty, and restenosis after coronary artery bypass surgery; peripheral vascular disease;

The invention furthermore relates to a pharmaceutical composition for the prevention of, delay of progression of, treatment of a disease or condition selected from the group consisting of

(a) type 2 diabetes mellitus and related diseases, disorders or conditions (including but not limited to diabetic nephropathy, diabetic retinopathy and diabetic neuropathy);

(b) insulin resistance and syndrome X, obesity and related diseases, disorders or conditions (including but not limited to not limited to Insulin Resistance, Type 2 Diabetes Mellitus, Reproductive Disorders, Cardiovascular Disease, Pulmonary Disease, Gallstones and Fasting-induced cholecystitis, Cancers and Cutaneous Disease, Cushing's Syndrome, Hypothyroidism, Insulinoma, Craniopharyngioma and Other Disorders Involving the Hypothalamus);

(c) hypertension including hypertension in the elderly, familial dyslipidemic hypertension, and isolated systolic hypertension (ISH); increased collagen formation, fibrosis, and remodeling following hypertension (antiproliferative effect of the combination); erectile dysfunction, impaired vascular compliance, stroke; all these diseases or conditions associated with or without hypertension;

(d) congestive heart failure, left ventricular hypertrophy, survival post myocardial infarction (MI), coronary artery diseases, atherosclerosis, angina pectoris, thrombosis;

(e) renal failure, especially chronic renal failure, glomerulosclerosis, nephropathy;

(f) hypothyroidism;

(g) endothelial dysfunction with or without hypertension;

(h) hyperlipidemia, hyperlipoproteinemia, hypertryglyceridemia, and hypercholesterolemia;

(i) macular degeneration, cataract, glaucoma;

(j) skin and connective tissue disorders, and

(k) restenosis after percutaneous transluminal angioplasty, and restenosis after coronary artery bypass surgery; peripheral vascular disease; comprising a combination of a DPP IV inhibitor or a pharmaceutically acceptable salt thereof with at least one therapeutic agent selected from the group consisting of consisting of

i) an antiobesity agent or a pharmaceutically acceptable salt thereof,

ii) an appetite regulating agent or a pharmaceutically acceptable salt thereof; and at least one additional pharmaceutically acceptable carrier.

Method or use as described above, wherein the disease or condition is selected from impaired glucose metabolism, conditions of impaired glucose tolerance, conditions of impaired fasting plasma glucose, obesity, diabetic retinopathy, diabetic nephropathy, diabetic neuropathy and foot ulcerations.

Method or use as described above, wherein the disease or condition is selected from diabetes preferably type 2 diabetes, IGT or obesity and diseases or conditions associated with diabetes or obesity.

Method or use as described above, wherein the disorders or conditions related to diabetes, particularly type 2 diabetes mellitus are selected from diabetic nephropathy, diabetic retinopathy and diabetic neuropathy, macular degeneration, coronary heart disease, myocardial infarction, diabetic cardiomyopathy, myocardial cell death, coronary artery diseases, peripheral arterial disease, stroke, limb ischemia, vascular restenosis, foot ulcerations, endothelial dysfunction and/or atherosclerosis.

Method or use as described above, wherein the DPP-IV inhibitor is administered simultaneously with the anti-obesity agent or appetite regulating agent or sequential in time with the anti-obesity agent or appetite regulating agent.

Method or use as described above, wherein the DPP-IV inhibitor and the anti-obesity agent or appetite regulating agent are administered in the form of a combination of the present invention such as a fixed combination or combined preparation or kit of part.

Combination, method or use as described herein, wherein the DPP-IV inhibitor is (S)-1-[(3-hydroxy-1-adamantyl)amino]acetyl-2-cyano-pyrrolidine and wherein the anti-obesity agent or appetite regulating agent is selected from the group consisting of phentermine, leptin, bromocriptine, dexamphetamine, amphetamine, fenfluramine, dexfenfluramine, sibutramine, orlistat, dexfenfluramine, mazindol, phentermine, phendimetrazine, diethylpropion, fluoxetine, bupropion, topiramate, diethylpropion, benzphetamine, phenylpropanolamine or ecopipam, ephedrine or pseudoephedrine or, in each case, a pharmaceutically acceptable salt thereof.

Combination, method or use as described above, wherein the DPP-IV inhibitor is (S)-1-{2-[5-cyanopyridin-2yl)amino]ethyl-aminoacetyl)-2-cyano-pyrrolidine and wherein the anti-obesity agent or appetite regulating agent is selected from the group consisting of phentermine, leptin, bromocriptine, dexamphetamine, amphetamine, fenfluramine, dexfenfluramine, sibutramine, orlistat, dexfenfluramine, mazindol, phentermine, phendimetrazine, diethylpropion, fluoxetine, bupropion, topiramate, diethylpropion, benzphetamine, phenylpropanolamine or ecopipam, ephedrine or pseudoephedrine or, in each case, a pharmaceutically acceptable salt thereof.

According the invention, when the DPP-IV inhibitors, and the anti-obesity agents are administered together, such administration can be sequential in time or simultaneous with, the simultaneous method being generally preferred. For sequential administration, the DPP-IV inhibitor, and the anti-obesity agent can be administered in any order. It is generally preferred that such administration be oral. It is especially preferred that the administration be oral and simultaneous. However, if the subject being treated is unable to swallow, or oral absorption is otherwise impaired or undesirable, parenteral or transdermal administration will be appropriate. When the DPP-IV inhibitor, and the anti-obesity agent are administered sequentially, the administration of each can be by the same method or by different methods.

A further aspect of the present invention relates to the use of a combination as described herein for the cosmetic treatment of a mammal in order to effect a cosmetically beneficial loss of body weight.

The invention also relates to a method of improving the bodily appearance of a warm-blooded animal comprising administering to a warm-blooded animal, including man, in need thereof a jointly effective amount of a combination of a DPP IV inhibitor or a pharmaceutically acceptable salt thereof with at least one agent selected from the group consisting of

i) an antiobesity agent or a pharmaceutically acceptable salt thereof,

ii) an appetite regulating agent or a pharmaceutically acceptable salt thereof; and at least one additional pharmaceutically acceptable carrier.

A further aspect of the present invention is a kit for the prevention of, delay of progression of, treatment of a disease or condition according to the present invention comprising

(a) an amount of a DPP IV inhibitor or a pharmaceutically acceptable salt thereof in a first unit dosage form;

(b) an amount of at least one therapeutic agent selected from the group consisting of components (i) to (ii), or, in each case, where appropriate, a pharmaceutically acceptable salt thereof in a second etc. unit dosage form; and

(c) a container for containing said first, second etc. unit forms.

In a variation thereof, the present invention likewise relates to a “kit-of-parts”, for example, in the sense that the components to be combined according to the present invention can be dosed independently or by use of different fixed combinations with distinguished amounts of the components, i.e. simultaneously or at different time points. The parts of the kit of parts can then e.g. be administered simultaneously or chronologically staggered, that is at different time points and with equal or different time intervals for any part of the kit of parts. Preferably, the time intervals are chosen such that the effect on the treated disease or condition in the combined use of the parts is larger than the effect that would be obtained by use of only any one of the components.

The present invention thus also relates to a kit of parts comprising

(a) an amount of a DPP IV inhibitor or a pharmaceutically acceptable salt thereof in a first unit dosage form;

(b) an amount of at least one therapeutic agent selected from the group consisting of components (i) to (ii), or, in each case, where appropriate, a pharmaceutically acceptable salt thereof, in the form of two or three or more separate units of the components (a) to (b).

The invention furthermore relates to a commercial package comprising the combination according to the present invention together with instructions for simultaneous, separate or sequential use.

In a preferred embodiment, the (commercial) product is a commercial package comprising as active ingredients the combination according to the present invention (in the form of two or three or more separate units of the components (a) or (b)), together with instructions for its simultaneous, separate or sequential use, or any combination thereof, in the delay of progression or treatment of the diseases (a) to (k) as mentioned herein.

All the preferences mentioned herein apply to the combination, composition, use, method of treatment, “kit of parts” and commercial package of the invention.

These pharmaceutical preparations are for enteral, such as oral, and also rectal or parenteral, administration to homeotherms, with the preparations comprising the pharmacological active compound either alone or together with customary pharmaceutical auxiliary substances. For example, the pharmaceutical preparations consist of from about 0.1% to 90%, preferably of from about 1% to about 80%, of the active compound. Pharmaceutical preparations for enteral or parenteral, and also for ocular, administration are, for example, in unit dose forms, such as coated tablets, tablets, capsules or suppositories and also ampoules. These are prepared in a manner that is known per se, for example using conventional mixing, granulation, coating, solubulizing or lyophilizing processes. Thus, pharmaceutical preparations for oral use can be obtained by combining the active compound(s) with solid excipients, if desired granulating a mixture which has been obtained, and, if required or necessary, processing the mixture or granulate into tablets or coated tablet cores after having added suitable auxiliary substances.

The dosage of the active compound can depend on a variety of factors, such as mode of administration, homeothermic species, age and/or individual condition.

Preferred dosages for the active ingredients of the pharmaceutical combination according to the present invention are therapeutically effective dosages, especially those which are commercially available.

Normally, in the case of oral administration, an approximate daily dose of from about 1 mg to about 360 mg is to be estimated e.g. for a patient of approximately 75 kg in weight.

The dosage of the active compound can depend on a variety of factors, such as mode of administration, homeothermic species, age and/or individual condition.

The pharmaceutical preparation will be supplied in the form of suitable dosage unit form, for example, a capsule or tablet, and comprising an amount, being together with the further component(s) jointly effective, e.g. 50 mg of LAF237.

The pharmaceutical composition according to the present invention as described hereinbefore may be used for simultaneous use or sequential use in any order, for separate use or as a fixed combination.

Thus according to a further embodiment, a DPP-IV inhibitor, is administered with an anti-obesity agent preferably in the form of a fixed pharmaceutical composition comprising a pharmaceutically acceptable carrier, vehicle or diluent. Accordingly, a DPP-IV inhibitor of this invention, can be administered with an anti-obesity agent as a fixed combination, in any conventional oral, parenteral or transdermal dosage form.

The doses of DPP-IV inhibitor of formula (I) to be administered to warm-blooded animals, for example human beings, of, for example, approximately 70 kg body weight, especially the doses effective in the inhibition of the DPP-IV enzyme, e.g. in lowering blood pressure and/or in improving the symptoms of glaucoma, are from approximately 3 mg to approximately 3 g, preferably from approximately 10 mg to approximately 1 g, for example approximately from 20 mg to 200 mg, per person per day, divided preferably into 1 to 4 single doses which may, for example, be of the same size. Usually, children receive about half of the adult dose. The dose necessary for each individual can be monitored, for example by measuring the serum concentration of the active ingredient, and adjusted to an optimum level. Single doses comprise, for example, 10, 40 or 100 mg per adult patient.

The dosage of (S)-1-[(3-hydroxy-1-adamantyl)amino]acetyl-2-cyano-pyrrolidine is preferably between 10 and 150 mg daily, most preferably between 25 and 100 mg or 25 and 50 mg daily. Preferred examples of daily oral dosage are 25, 30, 35, 45, 50, 55 or 60 mg. The application of the active ingredient may occur up to three times a day, preferably one or two times a day.

The preferred herein mentioned antiobesity agents, will be supplied in the form of suitable dosage unit form, for example, a capsule or tablet, and comprising a therapeutically effective amount, e.g. from about 2 to about 120 mg, as already described herein and in the prior art. The application of the active ingredient may occur up to three times a day, preferably one or two times a day. The same preferred dosage are selected for the fixed combinations.

Corresponding doses may be taken, for example, in the morning, at mid-day or in the evening.

Furthermore, the applicant has discovered a particular regimen improving the treatment and/or prevention of diabetes especially type 2 diabetes, IGT or obesity and conditions associated with diabetes or obesity. Surprisingly, if the combination of the invention preferably the DPP-IV inhibitor is taken in connection with the meal, preferably shortly before or at the beginning of the meal, optionally during the meal or even shortly after. The regimen meal-related according to the present invention results in an unexpected lowering of the conditions associated with diabetes or obesity, particularly in patients with diabetes e.g. type 2 diabetes, or hypertension.

The meal-related regimen according to the present invention particularly results in an unexpected lowering of cardiovascular diseases in patients with diabetes particularly with type 2 diabetes or obesity.

Thus in a further aspect, the present invention relates to the use of a combination of the invention, for the manufacture of a medicament for the prevention, delay of progression or the treatment of diabetes especially type 2 diabetes, IGT or obesity and conditions associated with diabetes or obesity, wherein the DPP-IV inhibitor preferably (S)-1-[(3-hydroxy-1-adamantyl) amino]acetyl-2-cyano-pyrrolidine (LAF237) of formula (I) is to be administered in relation to meals.

The present invention relates furthermore to a method for the prevention, delay of progression or treatment of diabetes especially type 2 diabetes, IGT or obesity and conditions associated with diabetes or obesity comprising administering to a warm-blooded animal, including man, in need thereof, a therapeutically effective amount of a combination of the invention, the administration of the DPP-IV inhibitor preferably (S)-1-[(3-hydroxy-1-adamantyl)amino]acetyl-2-cyano-pyrrolidine (LAF237) of formula (I) being in relation to meals.

Use or method as described above for the treatment of a patient with diabetes particularly with type 2 diabetes.

The designation “meal” as used in the present text is intended to mean breakfast, lunch dinner or midnight snack.

When the expression “meal-related” is used in the present text in connection with the administration of a DPP-IV inhibitor it preferably designates that the DPP-IV inhibitor is administered shortly before or at the beginning of the meal. However, the administration can obviously also take place during the meal or even shortly after without deviating from the idea behind the invention. Thus, the expression “meal-related” preferably means from about 30 preferably 10 minutes before the meal starts to about 10 minutes after the meal is finished, more preferred from about 5 minutes before the meal starts until the meal is finished, most preferred at the beginning of the meal.

Preferred DPP-IV inhibitors for the combinations, uses, methods, Kit of Parts of the present invention are 1-{2-[(5-cyanopyridin-2-yl) amino]ethylamino}acetyl-2 (S)-cyano-pyrrolidine dihydrochloride (DPP728), especially the dihydrochloride thereof, and (S)-1-[(3-hydroxy-1-adamantyl)amino]acetyl-2-cyano-pyrrolidine (LAF237), and L-threo-isoleucyl thiazolidine (compound code according to Probiodrug: P32/98 as described above), MK-0431, GSK23A, BMS-477118, 3(aminomethyl)-2-isobuthyl-1-oxo-4-phenyl-1,2-dihydro-6-isoquinolinecarboxamide and 2-{[3(aminomethyl)-2-isobuthyl-4-phenyl-1-oxo-1,2-dihydro-6-isoquinolyl]oxy}acetamide and optionally in any case pharmaceutical salts thereof.

Preferably, in case of free combinations, preferred are those dosages for launched products that have been approved and that have been marketed.

Especially preferred are low dose combinations.

The following examples can be carried out with the claimed combinations to show their claimed activity and unexpected effect.

Experience 1:

Animals and Surgery

All procedures involving animals is performed in accordance with the standards of the US Department of Health and Human Services and is approved by the Novartis Animal Care and Use Committee. Male obese Zucker rats (Charles River, Wilmington, Mass.) are individually housed under a reverse light cycle (lights on 2000 hours to 0800 hours) with free access to tap water and standard rodent chow (Purina Labs, Richmond, Ind.). At 11 weeks of age, the animals are aseptically implanted with a silastic catheter in the right jugular vein under Ketamine/Rompun/Acepromazin anaesthesia. The catheter is externalized in the nape of the neck and is filled with a solution of heparin and polyvinylpyrrolidone. The rats are allowed to recover from the surgery before the experiments.

Study Protocol and Measurements

At 12 weeks of age, the animals are orally dosed with vehicle (0.5% CMC) or test compounds for three weeks. On the 22^{nd} day, an oral glucose tolerance test is performed. Briefly, the rats are fasted approximately 16 hours. At −30 min time point on the day of the experiments, the animals are orally dosed with vehicle, test compounds alone (DPP-IV inhibitor e.g. LAF237 at 10 μmole/kg or the antiobesity agent e.g. at 10 mg/kg) or the combination. The cannulas are then connected to sampling tubing. At −10 and 0 min two basal samples (500 ul) are withdrawn. Glucose (1 g/kg) has to be given by gavage after the second sample. Additional samples are withdrawn at 5, 10, 15, 20, 30, 45, 60, 75, 90, and 120 min. All samples are replaced by donor blood from untreated rats containing citrate and sodium-citrate as anticoagulant. Blood samples are collected in chilled Eppendorf tubes containing EDTA and 100 KIU trasylol per ml of blood. Samples are then centrifuged and plasma are stored at −20° C. until analyses. On the 29^{th} day, an intralipid challenge test is performed. Briefly, the rats are fasted approximately 2 hours. At −30 min time point on the day of the experiments, the animals are orally dosed with vehicle, test compounds alone (LAF237 at 10 μmole/kg or an antiobesity compound at e.g. 10 mg/kg) or the combination. The cannulas are then connected to sampling tubing. At −10 and 0 min two basal samples (500 μl) are withdrawn. Intralipid fat emulsion (Fisher Scientific Inc, Pittsburg, Pa.) are given by gavage at 2 g/kg after the second sample. Additional samples are withdrawn at 5, 10, 15, 20, 30, 45, 60, 75, 90, and 120 min. All samples are replaced by donor blood from untreated rats containing citrate and sodium-citrate as anticoagulant. Blood samples are collected in chilled Eppendorf tubes containing EDTA and 100 KIU trasylol per ml of blood. Samples are centrifuged and plasma is stored at −20° C. until analyses.

Plasma glucose is analyzed using a modified Sigma Diagnostics glucose oxidase kit (Sigma Chemical Co, St. Louis, Mo.). Plasma immunoreactive insulin (IRI) concentration is assayed by a double antibody from Linco Research (St. Louis, Mo.). The assay has a lower limit of detection of 30 pmol/l with intra-assay and inter-assay variations of less than 5%. Plasma DPP-IV activity in plasma sample is measured as previously described [(Balken, et al 1999)]. Plasma levels of GLP-1 (7-36) amides are measured using he GLP-1 (active) Elisa Kit (Linco Research Cat#EGLP-35K, St. Louis, Mo.) [(Balkan, et al 1999)]. Plasma total cholesterol, triglyceride and free fatty acids levels is determined enzymatically with assay kits from Sigma Chemical Co (St. Louis, Mo.).

Results:

Effect of Combination Therapy of LAF237 and an Antiobesity Agent on Body Weight Gain in Zucker fa/fa Rats.

Rats treated with a DPP-IV inhibitor and an antiobesity agent can show an unexpected synergistic weight reduction compared to the rats treated with either agent given alone.

Effect of Combination Therapy of LAF237 and an Antiobesity Agent on OGTT Glucose or Insulin Excursions in Zucker fa/fa Rats.

An unexpected synergistic effect can be observed with our combinations.

Effect of Combination Therapy of LAF237 and an Antiobesity Agent on Plasma Fibrinogen in Zucker fa/fa Rats.

An unexpected synergistic effect can be observed with our combinations.

1. A combination comprising a DPP IV inhibitor or a pharmaceutically acceptable salt thereof, and at least one therapeutic agent selected from the group consisting of
i) an antiobesity agent or a pharmaceutically acceptable salt thereof,
ii) an appetite regulating agent or a pharmaceutically acceptable salt thereof.

2. A pharmaceutical composition comprising the combination of claim 1 and a pharmaceutically acceptable carrier.

3. The combination according to claim 1, in the form of a combined preparation or a fixed combination.

4. The combination according to claim 1, wherein the DPP-IV inhibitor is selected from 1-{2-[(5-cyanopyridin-2-yl) amino]ethylamino}acetyl-2 (S)-cyano-pyrrolidine dihydrochloride, and (S)-1-[(3-hydroxy-1-adamantyl)amino]acetyl-2-cyano-pyrrolidine, L-threo-isoleucyl thiazolidine, MK-0431, GSK23A, BMS-477118, 3-(aminomethyl)-2-isobuthyl-1-oxo-4-phenyl-1,2-dihydro-6-isoquinolinecarboxamide and 2-{[3-(aminomethyl)-2-isobuthyl-4-phenyl-1-oxo-1,2-dihydro-6-isoquinolyl]oxy}acetamide, and optionally in any case, a pharmaceutical salts thereof.

5. The combination according to claim 1, wherein the DPP-IV inhibitor is (S)-1-{2-[5-cyanopyridin-2yl)amino]ethyl-aminoacetyl)-2-cyano-pyrrolidine or (S)-1-[(3-hydroxy-1-adamantyl)amino]acetyl-2-cyano-pyrrolidine.

6. The combination according to claim 1, wherein the anti-obesity agent or appetite regulating agent is selected from the group consisting of phentermine, leptin, bromocriptine, dexamphetamine, amphetamine, fenfluramine, dexfenfluramine, sibutramine, orlistat, dexfenfluramine, mazindol, phentermine, phendimetrazine, diethylpropion, fluoxetine, bupropion, topiramate, diethylpropion, benzphetamine, phenylpropanolamine or ecopipam, ephedrine or pseudoephedrine; or, in each case, a pharmaceutically acceptable salt thereof.

7. A method for the prevention of, delay of progression of, treatment of a disease or condition selected from the group consisting of
(a) type 2 diabetes mellitus and related diseases, disorders or conditions;
(b) insulin resistance and syndrome X, obesity and related diseases, disorders or conditions;
(c) hypertension including hypertension in the elderly, familial dyslipidemic hypertension, and isolated systolic hypertension (ISH); increased collagen formation, fibrosis, and remodeling following hypertension; erectile dysfunction, impaired vascular compliance, stroke; all these diseases or conditions associated with or without hypertension;
(d) congestive heart failure, left ventricular hypertrophy, survival post myocardial infarction (MI), coronary artery diseases, atherosclerosis, angina pectoris, thrombosis;
(e) renal failure, especially chronic renal failure, glomerulosclerosis, nephropathy;
(f) hypothyroidism;
(g) endothelial dysfunction with or without hypertension;
(h) hyperlipidemia, hyperlipoproteinemia, hypertryglyceridemia, and hypercholesterolemia;
(i) macular degeneration, cataract, glaucoma;
(j) skin and connective tissue disorders, and
(k) restenosis after percutaneous transluminal angioplasty, and restenosis after coronary artery bypass surgery; peripheral vascular disease;
comprising administering to a warm-blooded animal, including man, in need thereof a jointly effective amount of a combination of a DPP IV inhibitor or a pharmaceutically acceptable salt thereof with at least one therapeutic agent selected from the group consisting of
(i) an antiobesity agent or a pharmaceutically acceptable salt thereof,
(ii) an appetite regulating agent or a pharmaceutically acceptable salt thereof,
(iii) a renin inhibitor or a pharmaceutically acceptable salt thereof.

8. (canceled)

9. (canceled)

10. The method according to claim 7 wherein the DPP-IV inhibitor is selected from 1-{2-[(5-cyanopyridin-2-yl)amino]ethylamino}acetyl-2 (S)-cyano-pyrrolidine dihydrochloride, and (S)-1-[(3-hydroxy-1-adamantyl)amino]acetyl-2-cyano-pyrrolidine, L-threo-isoleucyl thiazolidine, MK-0431, GSK23A, BMS-477118, 3-(aminomethyl)-2-isobuthyl-1-oxo-4-phenyl-1,2-dihydro-6-isoquinolinecarboxamide and 2-{[3-(aminomethyl)-2-isobuthyl-4-phenyl-1-oxo-1,2-dihydro-6-isoquinolyl]oxy}acetamide, and optionally in any case, a pharmaceutical salts thereof.

11. The method according to claim 7 wherein the anti-obesity agent or appetite regulating agent is selected from the group consisting of phentermine, leptin, bromocriptine, dexamphetamine, amphetamine, fenfluramine, dexfenfluramine, sibutramine, orlistat, dexfenfluramine, mazindol, phentermine, phendimetrazine, diethylpropion, fluoxetine, bupropion, topiramate, diethylpropion, benzphetamine, phenylpropanolamine or ecopipam, ephedrine or pseudoephedrine; or, in any case, a pharmaceutically acceptable salt thereof.

12. The method according to claim 7, wherein the DPP-IV inhibitor is selected from 1-{2-[(5-cyanopyridin-2-yl)amino]ethylamino}acetyl-2 (S)-cyano-pyrrolidine dihydrochloride, and (S)-1-[(3-hydroxy-1-adamantyl)amino]acetyl-2-cyano-pyrrolidine, L-threo-isoleucyl thiazolidine, MK-0431, GSK23A, BMS-477118, 3-(aminomethyl)-2-isobuthyl-1-oxo-4-phenyl-1,2-dihydro-6-isoquinolinecarboxamide and 2-{[3-(aminomethyl)-2-isobuthyl-4-phenyl-1-oxo-1,2-dihydro-6-isoquinolyl]oxy}acetamide, and wherein the anti-obesity agent or appetite regulating agent is selected from the group consisting of phentermine, leptin, bromocriptine, dexamphetamine, amphetamine, fenfluramine, dexfenfluramine, sibutramine, orlistat, dexfenfluramine, mazindol, phentermine, phendimetrazine, diethylpropion, fluoxetine, bupropion, topiramate, diethylpropion, benzphetamine, phenylpropanolamine or ecopipam, ephedrine or pseudoephedrine;
or, in any case, a pharmaceutically acceptable salt thereof.

13. The combination according to claim 2, wherein the DPP-IV inhibitor is (S)-1-{2-[5-cyanopyridin-2yl)amino]ethyl-aminoacetyl)-2-cyano-pyrrolidine or (S)-1-[(3-hydroxy-1-adamantyl)amino]acetyl-2-cyano-pyrrolidine, and wherein the anti-obesity agent or appetite regulating agent is selected from the group consisting of phentermine, leptin, bromocriptine, dexamphetamine, amphetamine, fenfluramine, dexfenfluramine, sibutramine, orlistat, dexfenfluramine, mazindol, phentermine, phendimetrazine, diethylpropion, fluoxetine, bupropion, topiramate, diethylpropion, benzphetamine, phenylpropanolamine or ecopipam, ephedrine or pseudoephedrine;
or, in any case, a pharmaceutically acceptable salt thereof.

14. (canceled)

15. The combination according to claim 1, wherein the DPP-IV inhibitor is (S)-1-{2-[5-cyanopyridin-2yl)amino]ethyl-aminoacetyl)-2-cyano-pyrrolidine or (S)-1-[(3-hydroxy-1-adamantyl)amino]acetyl-2-cyano-pyrrolidine, and wherein the anti-obesity agent or appetite regulating agent is selected from the group consisting of orlistat, sibutramine, diethylpropion, phen-fen and phentermine, or a pharmaceutically acceptable salt thereof.

16. The method according to claim 7, wherein the disease or condition is selected from diabetes preferably type 2 diabetes, IGT or obesity and diseases or conditions associated with diabetes or obesity.
